(19) United States
(12) Patent Application Publication
(10) Pub. No.: US 2003/0147814 A1
(43) Pub. Date: Aug. 7, 2003

(54) Medicinal aerosol formulations comprising ion pair complexes

(75) Inventors: Robert A. Scherrer, White Bear Lake, MN (US); James S. Stefely, Woodbury, MN (US); Stephen W. Stein, Lino Lakes, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/327,201

(22) Filed: Dec. 20, 2002

Related U.S. Application Data
(60) Provisional application No. 60/342,913, filed on Dec. 21, 2001.

(51) Int. Cl. A61L 9/04

(52) U.S. Cl. 424/45

(57) ABSTRACT

The invention comprises hydrofluorocarbon medicinal aerosol solutions comprising an ion pair complex comprising a drug and an excipient. The invention also relates to methods for delivering a highly respirable dose to a mammal with a medicinal aerosol comprising an ion pair complex.

This application claims benefit of priority to provisional patent application No. 60/342,913, filed Dec. 21, 2001.

### FIELD

The present invention relates to hydrofluorocarbon medicinal aerosol formulations, and, in particular, to medicinal aerosol formulations for use in metered dose inhaler (oral or nasal) products.

### BACKGROUND OF THE INVENTION

It is known that solution based metered dose inhalers can be used to treat a variety of medical conditions, although in the past most marketed solution formulations had significant drawbacks in terms of physicochemical stability and poor respirable fraction. Use of hydrofluorocarbon (HFC) propellants has become one of the known alternatives to avoid using CFCs due to environmental considerations, and medicinal aerosol solution formulations based on HFC propellants are known. A fundamental limitation, however, of most medicinal aerosol solutions is the amount of drug that can be dissolved in the formulation. It is often the case that the drug solubility in propellant alone is not sufficiently high to allow for practical delivery of a therapeutic dose. Modifications may be made to the formulation, for example the addition of polar cosolvents, which can increase the total drug solubility in a given formulation. Addition of polar cosolvents, particularly in relatively large amounts, can have certain disadvantages, however, including reduction of respirable fraction, unpleasant taste, and/or increased drug deposition in the naso-oropharyngeal cavity.

### SUMMARY OF THE INVENTION

It has now been found that certain compounds can be used to form ion pair complexes in solution formulations using HFC propellants. It has also now been found more generally that solution formulations having relatively high drug concentration and that avoid substantial amounts of low-volatility cosolvent can deliver exceptionally high respirable doses of drug. This can be achieved in various ways, such as where the drug is inherently highly soluble in the propellant formulation, but using an ion pair complex as set forth herein is a particularly good approach because it applies broadly to many drugs that are otherwise not sufficiently soluble.

Hence, in one aspect, the present invention thus provides a medicinal aerosol formulation comprising a hydrofluorocarbon propellant and an ion pair complex comprising a drug and an excipient compound in solution. The excipient is a compound of the structure R₁—X;

wherein R₁ is a linear, branched, or cyclic hydrocarbon with 1 to 26 carbons, which may be optionally interrupted by a —O—, —S—, or —N(R₄)— group;

X is selected from the group consisting of: —C(O)OH; —S(O₂)OH; —OS(O₂)OH; —OP(OH)₂O; —P(OH)₂O and —N(R₄)(R₄); and

R₄ is hydrogen or a linear, branched, or cyclic hydrocarbon with 1 to 18 carbons; and further wherein the propellant and ion pair complex form a solution.

In a preferred embodiment, the invention comprises a solution of propellant and cosolvent, wherein the ion pair complex is substantially more soluble in the propellant and cosolvent solution than the drug alone is soluble in the propellant and cosolvent solution. This can permit the use of a smaller amount of cosolvent or, conversely, a larger amount of drug.

The invention also relates to medicinal aerosol solution formulation products comprising ion pair complexes, which products may be equipped with a metered dose valve.

In another aspect, the invention comprises a method of delivering a medicinal aerosol to a mammal. A drug with a solubility in propellant alone of less than 0.5% by weight is provided. A medicinal solution is prepared comprising a mixture of a hydrofluorocarbon propellant and an ion pair complex comprising the drug and an excipient. A metered dose inhaler is prepared comprising the medicinal solution, a canister, and an actuator. The metered dose inhaler is placed in fluid communication with the oral cavity of a mammal, and a respirable dose of greater than 30 micrograms of the drug is delivered to the mammal with a single actuation of the metered dose inhaler.

Among the benefits of the present invention can be improved drug solubility, respirable mass, respirable fraction, and/or sustained drug release, without some or all of the disadvantages that can be caused by use of relatively large amounts of polar cosolvents.

### DETAILED DESCRIPTION OF THE INVENTION

The present invention comprises a medicinal aerosol formulation comprising a hydrofluorocarbon propellant and an ion pair complex comprising a drug and an excipient, wherein the propellant and ion pair complex form a solution. The excipient is a compound of the structure R₁—X.

Suitable propellants include hydrofluorocarbons (HFCs), such as 1,1,1,2-tetrafluoroethane (also referred to as propellant 134a, HFC-134a, or HFA-134a) and 1,1,1,2,3,3,3-heptafluoropropane (also referred to as propellant 227, HFC-227, or HFA-227), or mixtures of any of the foregoing. The propellant is preferably present in an amount sufficient to propel a plurality of doses of the drug from an aerosol canister, preferably a metered dose inhaler.

Suitable excipients comprise compounds of the structure R₁—X.

R₁ is a linear, branched, or cyclic hydrocarbon with 1 to 26 carbons, which may be optionally interrupted by a —O—, —S—, or —N(R₄)— group. Preferably, R₁ is a linear, branched, or cyclic hydrocarbon with 1 to 20 carbons, more preferably 6 to 20 carbons, and most preferably 12 to 18 carbons.

X is selected from the group consisting of: —C(O)OH; —S(O₂)OH; —OS(O₂)OH; —OP(OH)₂O; —P(OH)₂O and —N(R₄)(R₄) and is more preferably —C(O)OH.

R₄ is hydrogen or a linear, branched, or cyclic hydrocarbon with 1 to 18 carbons, more preferably 1 to 6 carbons, and most preferably 1 to 2 carbons.

Medicinal formulations according to the present invention contain a drug either dispersed or dissolved in the formulation in a therapeutically effective amount. As used herein the term “therapeutically effective amount” means an amount sufficient to induce a therapeutic effect, such as bronchodilation or antiviral activity. The amount will vary according to factors known to those skilled in the art, such as the pharmacological activity of the particular drug, the condition being treated, the frequency of administration, the treatment site, and any other therapeutic agents being coadministered.

As used herein, the term “drug,” includes its equivalents, “bioactive agent,” and “medicament” and is intended to have its broadest meaning as including substances intended for use in the diagnosis, cure, mitigation, treatment or prevention of disease, or to affect the structure or function of the body. The drugs must be capable of forming an ion pair. Preferably, they are suitable for oral and/or nasal inhalation. Delivery to the respiratory tract and/or lung, in order to effect bronchodilation and to treat conditions such as asthma and chronic obstructive pulmonary disease, is preferably by oral inhalation. Alternatively, to treat conditions such as rhinitis or allergic rhinitis, delivery is preferably by nasal inhalation.

Suitable drugs include, for example, antiallergics, anticancer agents, antifungals, antineoplastic agents, analgesics, bronchodilators, antihistamines, antiviral agents, antitussives, anginal preparations, antibiotics, anti-inflammatories, immunomodulators, 5-lipoxygenase inhibitors, leukotriene antagonists, phospholipase A₂ inhibitors, phosphodiesterase IV inhibitors, peptides, proteins, and vaccine preparations. Preferably, medicinal formulations according to the present invention include a drug in an amount and in a form such that the drug can be administered as an aerosol. More preferably, the drug is present in an amount such that the drug can produce its desired therapeutic effect with one dose from a conventional aerosol canister with a conventional valve, such as a metered dose valve. As used herein, an “amount” of the drug can be referred to in terms of quantity or concentration. A therapeutically effective amount of a drug can vary according to a variety of factors, such as the potency of the particular drug, the route of administration of the formulation, the mode of administration of the formulation, and the mechanical system used to administer the formulation. A therapeutically effective amount of a particular drug can be selected by those of ordinary skill in the art with consideration of such factors. Generally, a therapeutically effective amount will be from about 0.02 parts to about 2 parts, more preferably from about 0.1 parts to about 1 part, by weight based on 100 parts of the medicinal formulation.

The drug and excipient comprise an ion pair complex.

In a preferred embodiment, the drug is substantially insoluble in the propellant alone, but becomes substantially more soluble in association with the excipient.

It should be appreciated that by substantially insoluble it is not meant that the drug is utterly insoluble in the propellant. Rather, it is meant that the direct solubility of the drug in the propellant is quite limited and that it would be desirable to dissolve an amount of the drug over and above that amount which is directly soluble. That desired additional amount is not soluble in the propellant alone. This is often the case for a drug that is only slightly soluble in a propellant, when it may be desirable to dissolve more of the drug into the propellant than is possible by direct dissolution. According to the present invention, when the drug is combined with the excipient, the solubility of the drug in the propellant may be increased substantially. In particular, use of a polar cosolvent in conjunction with the excipient can substantially increase the solubility of drug in the medicinal formulation. Conversely, when a polar cosolvent is used to increase the solubility of drug in a propellant and cosolvent mixture, the amount of cosolvent necessary to solubilize the drug may be reduced substantially by combining the drug with the excipient forming an ion pair.

With the present invention, the drug and the excipient are in a true, homogeneous solution in the propellant. By a true, homogeneous solution, it is meant that the drug, the excipient and the propellant form a single liquid phase. The present invention is, therefore, distinguishable from the preparation of emulsions, micellar systems, and other colloidal suspensions that comprise at least two distinct phases, with one phase being dispersed within the other phase.

To assist in the understanding of the present invention, but not to be bound by theory, it is believed that the drug and the excipient are associated in the form of a complex between the excipient and the drug. Preferably, the excipient and the drug have oppositely charged ionic portions which associate to form an ion pair (IP) complex. Preferably, the drug comprises a cationic portion that associates with an anionic portion of the excipient. In the case where the excipient has a hydrophobic portion that does not interact with the drug, the ion pair complex can be referred to as a hydrophobic ion pair (HIP) complex. Hydrophobic ion pair (HIP) complexes will be understood by one skilled in the art and are described, for example, in U.S. Pat. No. 5,770,559 (Manning, et al.), the disclosure of which is incorporated herein by reference.

Medicinal formulations according to the present invention can include an optional cosolvent or mixtures of cosolvents. The cosolvent is typically used in an amount effective to dissolve the ion pair complex. Preferably, the cosolvent is used in an amount of about 0.01 to about 25% by weight, more preferably about 0.01 to about 15%, and most preferably about 0.01 to about 6%, based on the total weight of the formulation. Examples of suitable cosolvents include ethanol, isopropanol, acetone, ethyl lactate, dimethyl ether, menthol, tetrahydrofuran, and ethyl acetate. Ethanol is a preferred cosolvent.

Other additives (i.e., excipients), such as lubricants, surfactants, and taste masking ingredients, can also be included in medicinal formulations of the present invention.

The amount of excipient used will depend upon a number of factors, including the type and amount of drug used and the desired therapeutic effect. In a preferred embodiment the molar ratio of excipient to drug will be between about 1.5:1 and 1:2, and more preferably will be about 1:1.

The ability of an MDI to deliver drug particles to the lung depends on the ability of the MDI to generate particles in the respirable size range. Smaller particles generally are more able to penetrate through the oropharynx and be delivered to the lung. As defined herein, particles with an aerodynamic diameter smaller than about 4.7 micrometers are defined as “respirable” and particles larger than this size are defined as “non-respirable”. The “respirable dose” delivered by an MDI is the weight of drug associated with particles smaller than about 4.7 micrometers delivered by the MDI with each actuation. The “respirable fraction” of an MDI is the respirable dose divided by the total weight of drug delivered from the MDI actuator with each actuation. The “respirable efficiency” of an MDI is the respirable dose divided by the total weight of drug delivered from the MDI valve with each actuation.

It has been found that a combination of factors must be met to provide a solution MDI with a relatively high respirable dose. The MDI must have a relatively high concentration of drug in the solution and it also must be relatively efficient at delivering the drug in a respirable form. Many drugs are poorly soluble in HFA propellant alone, and it is often necessary to include large amounts of polar cosolvents in a formulation in order to dissolve drug in an amount sufficient to provide a therapeutic effect. This can result in a sufficient drug concentration, but a lower respirable dose due to the large amount of polar cosolvent. In one aspect where the present invention has particular utility, the drug has a solubility in propellant alone of less than about 0.5% by weight, preferably less than 0.2% by weight, and most preferably less than 0.1% by weight. Increasing the amount of typical polar cosolvents, such as ethanol, can increase the concentration of drug dissolvable in a solution MDI, but will also decrease the respirable efficiency of the MDI. Thus, a potential increase in respirable dose due to increased drug concentration due to cosolvent is limited by a corresponding reduction in respirable efficiency.

The respirable dose delivered to a mammal with a single actuation of a metered dose inhaler in this aspect of the invention is greater than 30 micrograms, preferably greater than 50 micrograms, and more preferably greater than 100 micrograms. It is also preferred that the delivered dose have a high respirable fraction, which is the percentage of the total dose delivered that is respirable. The respirable fraction is preferably greater than 40% and most preferably greater than 60%. In one aspect of the invention, for an ion pair formulation at a selected cosolvent level, the respirable dose of the drug delivered to a mammal with a single actuation of a metered dose inhaler is greater than that achieved with cosolvent alone (i.e., in the absence of the ion pair).

Conventional aerosol canisters, such as those of aluminum, glass, stainless steel, or polyethylene terephthalate, can be used to contain the medicinal formulations according to the present invention. Aerosol canisters equipped with conventional valves, preferably, metered dose valves, can be used to deliver the formulations of the invention. The selection of the appropriate valve assembly typically depends on the components in the medicinal formulation.

Preparation of the compositions may be by a variety of conventional methods. Where a cosolvent is used, a mixture of drug, excipient, and cosolvent may be prepared, to which propellant is subsequently added to prepare a metered dose inhaler.

### EXAMPLES

Examples 1-16 show how very high efficiency solution formulations can be made using previously disclosed excipients, such as oligolactic acids and polyethylene glycol.

Examples 17-30 show ion pair formulations using particular types of excipients.

### Examples 1 to 3

Albuterol base, acetylated oligolactic (OLA) acid with an average chain length of 10.5, and ethanol were added to a canister which was capped with a continuous valve. HFA-134a propellant was added to the canister and mixed together to form a solution formulation. The relative weight percentages of each component are shown below in table 1. The contents of the canister were chilled and transferred to a 15 mL canister and a 50 microliter Spraymiser (trademark of 3M Co.) valve was crimped onto the canister. The canister was placed into a standard solution MDI actuator with an orifice diameter of approximately 0.30 mm.

Inertial impactor measurements were conducted using the U.S. Pharmacopeia recommended inlet (as described in U.S. Pat. No. 1995, Section 601, Aerosols, p.1764) and an impactor with a 4.7 micron cutpoint stage. Tests were conducted by actuating the MDIs five times into the inertial impactor apparatus operating at a flowrate of 28.3 liters per minute. The drug depositing on each component of the inertial impactor apparatus were determined by rinsing the component with a solution of 55 parts 0.1% o-phosphoric acid: 45 parts methanol (v/v) to dissolve the drug and then determining the concentration of drug in that solution using reverse phase HPLC with external standard quantitation and UV detection at 225 nm. The respirable mass is defined as the weight of drug (in micrograms) per actuation that deposited on all components of the impactor apparatus beyond the 4.7 micron cutpoint stage. Respirable mass results are shown below in table 1.

TABLE 1

| | Albuterol | | | | |
|---|---|---|---|---|---|
| Example | Base | OLA | Ethanol | HFA-134a | Resp. Mass |
| Number | (% w/w) | (% w/w) | (% w/w) | (% w/w) | (mcg/act) |
| 1 | 0.32 | 1.6 | 2.0 | 96.1 | 79.9 |
| 2 | 0.31 | 1.0 | 4.0 | 94.7 | 71.2 |
| 3 | 0.30 | 0.9 | 8.0 | 90.8 | 45.3 |

### Examples 4 to 8

A drug (4-amino-2-ethoxymethyl-α,α-dimethyl-1H-imidazo[4,5-c]quinoline-1-ethanol), oligolactic (OLA) acid with an average chain length of 10.5, and ethanol were added to a canister which was capped with a continuous valve. HFA-134a propellant was added to the canister and mixed together to form a solution formulation. The relative weight percentages of each component are shown below in table 2. The contents of the canister were chilled and transferred to a 15 mL canister and a 50 microliter Spraymiser (trademark of 3M Co.) valve was crimped onto the canister. The canister was placed into a standard solution MDI actuator with an orifice diameter of approximately 0.30 mm.

Inertial impactor measurements were conducted using the U.S. Pharmacopeia recommended inlet (as described in U.S. Pat. No. 1995, Section 601, Aerosols, p.1764) and an impactor with a 4.7 micron cutpoint stage. Tests were conducted by actuating the MDIs five times into the inertial impactor apparatus operating at a flowrate of 28.3 liters per minute. The drug depositing on each component of the inertial impactor apparatus were determined by rinsing the component with methanol to dissolve the drug and then determining the concentration of drug in that solution using an ion exchange HPLC method with external standard quantitation and UV detection at 247 nm. The respirable mass is defined as the weight of drug (in micrograms) per actuation that deposited on all components of the impactor apparatus beyond the 4.7 micron cutpoint stage. Respirable mass results are shown below in table 2.

TABLE 2

| Example | Drug | OLA | Ethanol | HFA-134a | Resp. Mass |
|---|---|---|---|---|---|
| Number | (% w/w) | (% w/w) | (% w/w) | (% w/w) | (mcg/act) |
| 4 | 0.31 | 1.6 | 1 | 97.1 | 100.4 |
| 5 | 0.31 | 1.6 | 3 | 95.1 | 87.5 |
| 6 | 0.31 | 1.6 | 6 | 92.1 | 70.5 |
| 7 | 0.31 | 1.5 | 10 | 88.2 | 53.0 |
| 8 | 0.31 | 1.2 | 15 | 83.5 | 38.9 |

### Examples 9 to 16

A drug (4-amino-2-ethoxymethyl-α,α-dimethyl-1H-imidazo[4,5-c]quinoline-1-ethanol), carboxylic acid functionalized polyethyleneglycol with a molecular weight of 350 and ethanol were added to a canister which was capped with a continuous valve. The carboxylic acid functionalized polyethyleneglycol was prepared by treating non-functionalized polyethyleneglycol with t-butoxide followed by the addition of ethyl bromylacetate, and subsequent hydrolysis with HCI. HFA-134a propellant was added to the canister and mixed together to form a solution formulation. The relative weight percentages of each component are shown below in table 3. The contents of the canister were chilled and transferred to a 15 mL canister and a 50 microliter Spraymiser (trademark of 3M Co.) valve was crimped onto the canister. The canister was placed into a standard solution MDI actuator with an orifice diameter of approximately 0.30 mm.

Inertial impactor measurements were conducted using the U.S. Pharmacopeia recommended inlet (as described in U.S. Pat. No. 1995, Section 601, Aerosols, p.1764) and an impactor with a 4.7 micron cutpoint stage. Tests were conducted by actuating the MDIs five times into the inertial impactor apparatus operating at a flowrate of 28.3 liters per minute. The drug depositing on each component of the inertial impactor apparatus were determined by rinsing the component with a solution of 1 part 10.0 M HCL: 99 parts, methanol (v:v) to dissolve the drug and then determining the concentration of drug in that solution using a UV absorbance method with detection at 321 nm. The respirable mass is defined as the weight of material (in micrograms) per actuation that deposited on all components of the impactor apparatus beyond the 4.7 micron cutpoint stage. Respirable fraction is defined as the weight percentage of the drug that is respirable divided by the total amount of drug delivered from the actuator. Respirable mass and respirable fraction results are shown below in table 3.

TABLE 3

| Example | Drug | PEG | Ethanol | HFA-134a | Resp. Mass | Resp. Fract. |
|---|---|---|---|---|---|---|
| Number | (% w/w) | (% w/w) | (% w/w) | (% w/w) | (mcg/act) | (%) |
| 9 | 0.16 | 0.5 | 2.2 | 97.1 | 80.7 | 73.8 |
| 10 | 0.26 | 0.5 | 5.4 | 93.8 | 98.8 | 60.9 |
| 11 | 0.33 | 0.5 | 10.6 | 88.5 | 86.0 | 42.2 |
| 12 | 0.30 | 1.1 | 2.1 | 96.5 | 147.5 | 68.0 |
| 13 | 0.40 | 1.1 | 5.3 | 93.2 | 150.3 | 59.4 |
| 14 | 0.56 | 1.1 | 10.6 | 87.8 | 130.6 | 41.5 |
| 15 | 0.82 | 2.1 | 5.3 | 91.8 | 240.4 | 45.9 |
| 16 | 1.03 | 2.1 | 10.6 | 86.3 | 171.8 | 30.0 |

### Example 17

A drug (4-amino-2-ethoxymethyl-α,α-dimethyl-1H-imidazo[4,5-c]quinoline-1-ethanol), lauric acid, and ethanol were added to a canister which was capped with a continuous valve. HFA-134a propellant was added to the canister and mixed together to form a solution formulation. The relative weight percentage of lauric acid was 0.99% and the relative weight percentage of ethanol was 14.85%. An excess of drug was provided. The canister was shaken for at least two days. A continuous valve was crimped onto a second, empty canister, which was chilled by being placed on dry ice. The formulation from the first canister was passed through a 0.22 micron filter, and into the second canister by depressing both continuous valves. The vapor pressure of the formulation in the first canister caused the formulation to flow through the filter. The solubility of the drug is taken as the concentration of drug in the solution that passes through the filter and into the second canister. The solution drug concentration was assayed for drug concentration using an ion exchange HPLC method with external standard quantitation and UV detection at 247 nm. The resulting drug solubility was 0.42%.

### Examples 18 to 20

A series of formulations was prepared following the method of example 17, with the exception that isostearic acid was used in place of lauric acid. The relative weight percentages of each component and the drug solubility are shown below in table 4.

TABLE 4

| | isostearic acid | Ethanol | Drug Solubility |
|---|---|---|---|
| Example Number | (% w/w) | (% w/w) | (% w/w) |
| 18 | 0.51 | 9.97 | 0.39 |
| 19 | 0.11 | 15.87 | 0.38 |
| 20 | 1.03 | 15.49 | 0.95 |

### Example 21 to 30

Albuterol base, an acyl acid, and ethanol were added to a canister which was capped with a continuous valve. HFA-134a propellant was added to the canister and mixed together to form a solution formulation. An excess of drug was provided. The canister was shaken for at least two days. A continuous valve was crimped onto a second, empty canister, which was chilled by being placed on dry ice. The formulation from the first canister was passed through a 0.22 micron filter, and into the second canister by depressing both continuous valves. The vapor pressure of the formulation in the first canister caused the formulation to flow through the filter and into the second canister. The solution in the second canister was assayed for drug concentration using a reverse phase HPLC with external standard quantitation and UV detection at 225 nm with a mobile phase of 55 parts 0.1% o-phosphoric acid: 45 parts methanol (v/v). The solubility of the drug in the formulation is taken as the concentration of drug in the solution that passes through the filter and into the second canister. The resulting drug solubilities are shown in Table 5.

### Comparative Example 1

A formulation was prepared following the description of examples 21 to 25, with the exception that no acyl acid was added to the formulation. The resulting drug solubility is shown in Table 5.

TABLE 5

| Example | Ethanol | | Acid | Drug Solubility |
|---|---|---|---|---|
| number | (% w/w) | Acid type | (% w/w) | (% w/w) |
| 21 | 10.0 | octanoic | 1.23 | 0.80 |
| 22 | 10.0 | decanoic | 1.03 | 0.73 |
| 23 | 10.0 | lauric | 1.04 | 0.48 |
| 24 | 10.0 | octanoic | 0.53 | 0.27 |
| 25 | 10.0 | lauric | 0.49 | 0.47 |
| 26 | 15.5 | octanoic | 1.27 | 2.28 |
| 27 | 15.7 | decanoic | 1.07 | 1.65 |
| 28 | 14.3 | palmitic | 0.94 | 0.63 |
| 29 | 15.7 | octanoic | 0.55 | 1.06 |
| 30 | 15.9 | decanoic | 0.51 | 0.89 |
| Comp. 1 | 10.0 | none | NA | 0.041 |

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow

We claim:

1. A medicinal aerosol formulation comprising:
a hydrofluorocarbon propellant;
an ion pair complex comprising a drug and an excipient, wherein the excipient is a compound of the structure R₁—X;
wherein R₁ is a linear, branched, or cyclic hydrocarbon with 1 to 26 carbons, which may be optionally interrupted by a —O—, —S—, or —N(R₄)— group;
X is selected from the group consisting of: —C(O)OH; —S(O₂)OH; —OS(O₂)OH; —OP(OH)₂O; —P(OH)₂O and —N(R₄)(R₄); and
R₄ is hydrogen or a linear, branched, or cyclic hydrocarbon with 1 to 18 carbons;
and further wherein the propellant and ion pair complex form a solution.

2. The medicinal aerosol solution formulation of claim 1 wherein the drug alone is not soluble in the propellant at a therapeutically effective concentration.

3. The medicinal aerosol solution formulation of claim 1 wherein the ion pair complex is substantially more soluble in the propellant than the drug alone is soluble in the propellant.

4. The medicinal aerosol solution formulation of claim 1 further comprising a polar cosolvent.

5. The medicinal aerosol solution formulation of claim 4 wherein the propellant and cosolvent form a solution.

6. The medicinal aerosol solution formulation of claim 5 wherein the ion pair complex is substantially more soluble in the propellant and cosolvent solution than the drug alone is soluble in the propellant and cosolvent solution.

7. The medicinal aerosol solution formulation of claim 1 wherein R₁ is a linear, branched, or cyclic hydrocarbon with 1 to 26 carbons.

8. The medicinal aerosol solution formulation of claim 1 wherein X is —C(O)OH.

9. A method of delivering a medicinal aerosol to a mammal comprising:
providing a drug with a solubility in propellant alone of less than 0.5% by weight;
preparing a medicinal solution comprising a mixture of a hydrofluorocarbon propellant and an ion pair complex comprising the drug and an excipient;
preparing a metered dose inhaler comprising the medicinal solution, a canister, and an actuator;
placing the metered dose inhaler in fluid communication with the oral cavity of a mammal; and
delivering a respirable dose of drug greater than 30 micrograms to the mammal with a single actuation of the metered dose inhaler.

10. The method of claim 9 wherein the medicinal solution further comprises a polar cosolvent.

11. The method of claim 9 wherein the respirable fraction of the delivered dose is greater than 40%.

12. The method of claim 9 wherein the respirable fraction of the delivered dose is greater than 60%.

13. The method of claim 9 wherein the delivered respirable dose is greater than 100 micrograms.

14. The method of claim 9 wherein the excipient consists essentially of a linear, branched, or cyclic hydrocarbon with an acid or amine end-group.

15. The method of claim 14 wherein the excipient is a compound of the structure R₁—X;
wherein R₁ is a linear, branched, or cyclic hydrocarbon with 1 to 26 carbons, which may be optionally interrupted by a —O—, —S—, or —N(R₄)— group;
X is selected from the group consisting of: —C(O)OH; —S(O₂)OH; —OS(O₂)OH; —OP(OH)₂O; —P(OH)₂O and —N(R₄)(R₄); and
R₄ is hydrogen or a linear, branched, or cyclic hydrocarbon with 1 to 18 carbons.

16. A medicinal aerosol solution formulation product comprising:
an aerosol container equipped with a dispensing valve;
an actuator; and
a medicinal aerosol formulation contained within the aerosol container;
wherein the medicinal aerosol formulation comprises:
a hydrofluorocarbon propellant;
an ion pair complex comprising a drug and an excipient;
wherein the drug has a solubility in propellant alone of less than 0.5% by weight, and further wherein the propellant and ion pair complex form a solution;
wherein the medicinal aerosol solution formulation product will deliver a respirable dose of drug greater than 30 micrograms when actuated.

17. The medicinal aerosol solution formulation product of claim 16 wherein the respirable dose delivered is greater than 50 micrograms.

18. The medicinal aerosol solution formulation product of claim 16 wherein the respirable dose delivered is greater than 100 micrograms.

19. The medicinal aerosol solution formulation product of claim 16 wherein the excipient is a compound of the structure R₁—X;
wherein R₁ is a linear, branched, or cyclic hydrocarbon with 1 to 26 carbons, which may be optionally interrupted by a —O—, —S—, or —N(R₄)— group;
X is selected from the group consisting of: —C(O)OH; —S(O₂)OH; —OS(O₂)OH; —OP(OH)₂O; —P(OH)₂O and —N(R₄)(R₄); and
R₄ is hydrogen or a linear, branched, or cyclic hydrocarbon with 1 to 18 carbons;
and further wherein the propellant and ion pair complex form a solution.

20. The medicinal aerosol solution formulation product of claim 16 wherein the medicinal solution further comprises a polar cosolvent.
